(19) United States
(12) Patent Application Publication
(10) Pub. No.: US 2011/0033338 A1
(43) Pub. Date: Feb. 10, 2011

(54) MICROFLUIDIC CIRCUIT ELEMENT COMPRISING MICROFLUIDIC CHANNEL WITH NANO INTERSTICES AND FABRICATION METHOD THEREOF

(75) Inventors: Sin Kil Cho, Seoul (KR); Seok Chung, Cambridge, MA (US)

Correspondence Address: SUGHRUE MION, PLLC, 2100 PENNSYLVANIA AVENUE, N.W., SUITE 800, WASHINGTON, DC, 20037, US

(73) Assignee: INCYTO CO., LTD., Cheonan-si, Chungcheongnam-do (unknown)

(21) Appl. No.: 12/936,839

(22) PCT Filed: Apr. 10, 2009
(86) PCT No.: PCT/KR09/01853

§ 371 Date: Oct. 7, 2010

(30) Foreign Application Priority Data
Apr. 11, 2008 (KR) 10-2008-0033757

(51) Int. Cl. G01N 33/48; B29C 65/08; B29C 65/14

(52) U.S. Cl. 422/68.1; 156/292; 156/272.6

(57) ABSTRACT

A microfluidic circuit element comprising a microfluidic channel, in which the microfluidic channel has nano interstices formed at both sides thereof and having a height less than that of the center of the channel, gives more driving force of the microfluidic channel and provides stable flow of a fluid.

### FIELD OF THE INVENTION

The present invention relates to a microfluidic circuit element having a microfluidic channel for stabilizing the flow of a fluid and a method for fabricating the same.

### BACKGROUND OF THE INVENTION

Microfluidics, which concerns the control of the flow and transfer of a very small amount of fluid, is essential for driving an apparatus for diagnosing and analyzing a sample, which may be executed using various driving methods such as a pressure-driven method using an applied pressure to a fluid injection portion; an electrophoretic method using an applied voltage across a microchannel; an electroosmotic method; and a capillary flow method using capillary force.

A typical example of a microfluidic device driven using a pressure-driven method is illustrated in U.S. Pat. No. 6,296,020, in which the cross-sectional area of a channel and the hydrophobicity of the channel are controlled with a passive valve installed in a hydrophobic fluidic circuit device. In addition, U.S. Pat. No. 6,637,463 discloses a microfluidic device in which channels having pressure gradients have been designed so that a fluid is uniformly distributed through the channels.

The capillary flow method, in particular, which uses capillary force spontaneously occurring in microchannels is advantageous because a very small amount of a fluid moves spontaneously and instantly along specific channels without the use of an additional driving means. Hence, extensive studies of microfluidic systems using such capillary flow method have been recently conducted. For example, U.S. Pat. No. 6,271,040 discloses a diagnostic biochip in which a sample is transferred using only the naturally-occuring capillary flow in microchannels without the use of a porous material, and the sample transferred in such a way was allowed to react with the biochips to detect a specific component in the sample. Also, U.S. Pat. No. 6,113,855 discloses a diagnostic apparatus in which hexagonal micro-pillars are appropriately arranged to generate capillary force for transferring a sample through the space between the pillars.

In order to achieve a satisfactory flow of a fluid in the conventional microfluidic device using the capillary flow method, the surface wettability of the capillary wall must be good. In case of a conventional plastic microfluidic device, such surface wettability of the plastic is unacceptably low, and to improve the poor surface wettability, a treatment, e.g., corona, surface coating and plasma treatments, has been conventionally used. For example, a method of roughening the inner surface of a microfluidic channel to enhance a fluid flow rate has been reported in WO 2007/075287.

However, the above methods for improving the wettability makes it difficult to achieve mass production of microfluidic devices, and they may also cause processing problems such as a need for the use of additional devices to carry out additional tasks. Further, because the effects of these treatment methods may deteriorate over a large period of use, it is difficult to maintain a constant, stable flow of a fluid.

### SUMMARY OF THE INVENTION

Accordingly, it is an object of the present invention to provide a microfluidic circuit element, which enables a fluid to flow only by the action of capillary force without an additional need for surface treatment such as chemical treatment or plasma treatment, is capable of maintaining the flow of a fluid uniform over a long period of time, and is easily fabricated without limitation of material. Another object of the present invention is to provide a method for fabricating said microfluidic circuit element.

In accordance with one aspect of the present invention, there is provided a microfluidic circuit element comprising a first substrate and a second substrate in a laminate form, the first substrate having a groove for defining a microfluidic channel which is formed on the side facing the second substrate and has an inlet and an outlet for a sample to flow through said channel, wherein the microfluidic channel has nano interstices formed at both sides thereof, the height of the nano interstices being less than that of the center of the microfluidic channel.

In accordance with another aspect of the present invention, there is provided a method for fabricating a microfluidic circuit element, comprising joining a first substrate and a second substrate such that a groove is formed therebetween to serve as a microfluidic channel which has an inlet and an outlet for a sample to flow therethrough and nano interstices having a height which is less than that of the center of the microfluidic channel formed at both sides of the microfluidic channel.

### BRIEF DESCRIPTION OF THE DRAWINGS

The above and other objects and features of the present invention will become apparent from the following description of the invention, when taken in conjunction with the accompanying drawings, which respectively show:

FIG. 1A: a schematic view of a conventional microfluidic channel;

FIG. 1B: a schematic view of the cross-section viewed along the dotted line A-A′ of FIG. 6 of a microfluidic channel according to an embodiment of the present invention;

FIG. 2A: a process of forming nano interstices joined by using a solvent, applying heat, a pressure or a laser beam according to the present invention;

FIG. 2B: a process of forming nano interstices joined by ultrasonic radiation according to the present invention;

FIG. 2C: a process of forming nano interstices joined using an adhesive or tape according to the present invention;

FIGS. 2D to 2G: various modifications of the nano interstices according to the present invention;

FIG. 3A: a process of forming a channel having nano interstices joined using solvent according to the present invention;

FIG. 3B: a process of forming a channel having no nano interstices by using laser-joining;

FIG. 4A: a cross-section at view of the microfluidic channel having nano interstices formed using a solvent according to the present invention, and SEM images corresponding thereto.

FIG. 4B: a cross-section at view of a microfluidic channel having no nano interstices formed by laser-joining, and SEM images corresponding thereto;

FIG. 5A: the fluid flowability through the microfluidic channel having no nano interstices formed by laser-joining;

FIG. 5B: the fluid flowability through the microfluidic channel having the nano interstices according to the present invention; and

FIG. 6: an example of the microfluidic circuit element according to an embodiment of the present invention and a photograph thereof.

### DETAILED DESCRIPTION OF THE INVENTION

The microfluidic circuit element according to an embodiment of the present invention comprises a first substrate and a second substrate on the first substrate, the first substrate having a groove for defining a microfluidic channel which is formed on the side facing the second substrate and has an inlet and an outlet for a sample to flow through said channel, wherein the microfluidic channel has nano interstices formed at both sides thereof, the height of the nano interstices being less than that of the center of the microfluidic channel.

The size of the microfluidic channel is not limited, but, the height of the microfluidic channel is preferably in the range of 2 μm to 5 mm, and the width of the microfluidic channel is preferably in the range of 2 μm to 5 mm or larger. The shape of the microfluidic channel may be of any kind. For example, the cross-section of the microfluidic channel may have a rectangular or other shape, e.g., a circular shape and a semicircular shape, provided that it is equipped with the above-mentioned nano interstices to realize the effects brought thereby.

The flow of a microfluid through the microfluidic channel may be driven by using pressure application, electrophoresis procedure, or capillary force. The use of capillary force is prefered because of the reasons that a fluid may be easily loaded or transferred, and a simplified device or system may be adopted without the need for external applied energy.

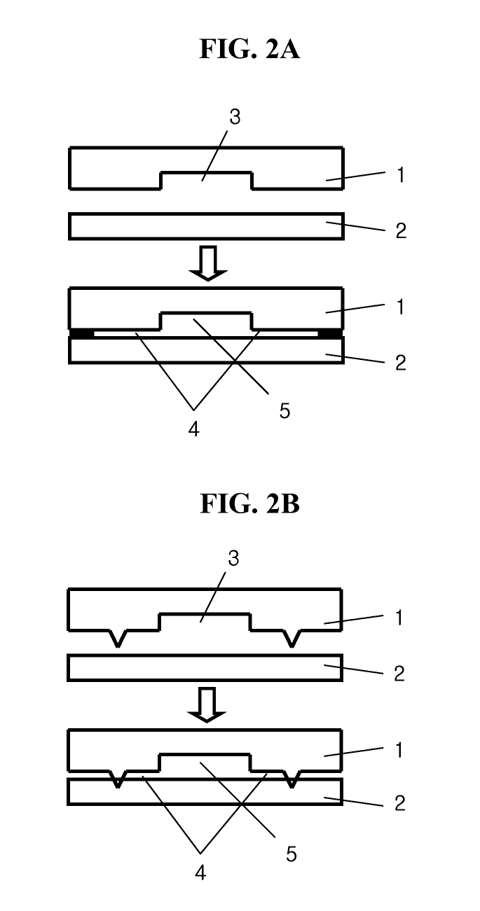

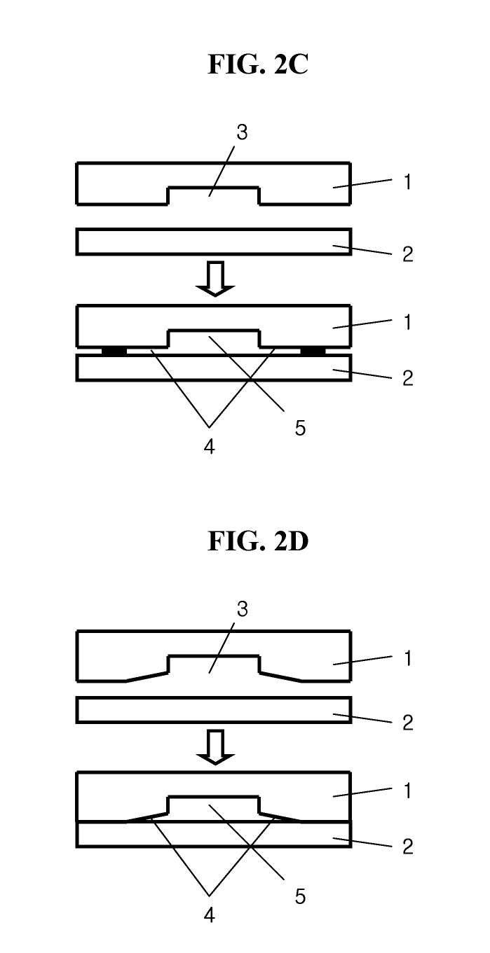

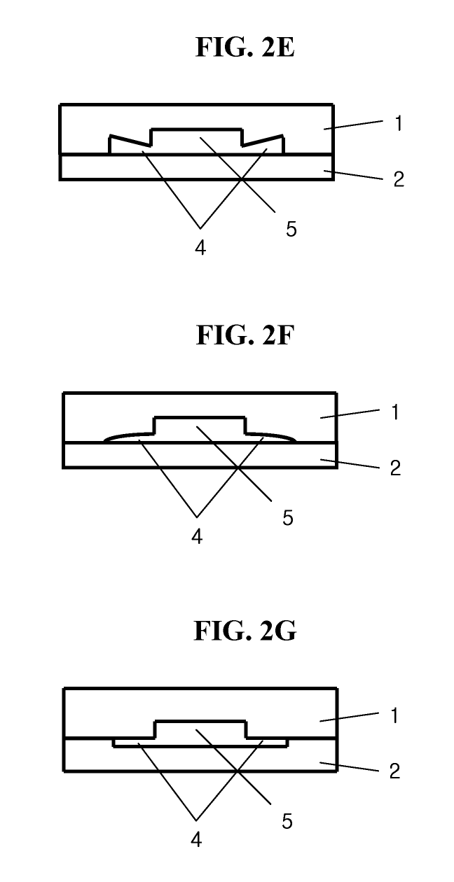

To maintain a stable flow of a sample fluid, the surface wettability is particularly important. For the improvement of the surface wettability, the cross-section of the nano interstices formed according to the present invention may be of a rectangular shape having a high aspect ratio, although, the present invention is not limited thereto: other shapes including irregular shapes may also be employed (see FIGS. 2D to 2G) The height of the nano interstices is preferably in the range of 10 nm to 5 μm, which facilitates the capillary flow of the fluid through the nano interstices to stabilize the overall flow through the channel.

The material for manufacturing microfluidic circuit element of the present invention may be any of those which enable the manufacture of the microfluidic system, and examples thereof are a silicon wafer, glass, pyrex, PDMS(polydimethylsiloxane), plastic, e.g., acryls, PMMA, PC, and others.

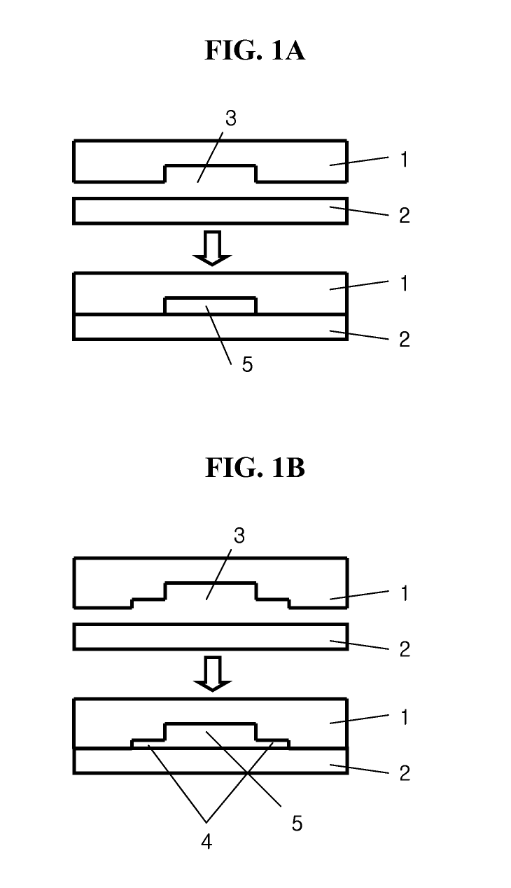

The present invention also provides a method for fabricating the microfluidic circuit element having nano interstices. Referring to FIG. 1B, the fabrication method comprises joining a first substrate (1) and a second substrate (2) such that a groove (3) is formed therebetween to serve as a microfluidic channel (5) which is provided with an inlet and an outlet (6) for a sample to flow therethrough and nano interstices (4) having a height which is less than that of the center of the microfluidic channel, said interstices formed at both sides of the microfluidic channel.

For the formation of the microfluidic channel, the first and second substrates (1 and 2) are washed, or may be subjected to any of the known surface treatment methods to make the surface of the channel hydrophilic. Before joining of the first and second substrates (1 and 2), a chemical treatment or an oxygen plasma treatment may be performed to increase the surface wettability of the surface of the channel and the interstices. In case an oxygen plasma treatment is performed, the surface is made hydrophilic by reducing the surface contact angle, but the effect of such treatment lasts only about three or four months.

Examples of the method for fabricating the microfluidic channel include silicon microprocessing, glass microprocessing, plastic microprocessing, and PDMS microprocessing. Among them, the glass microprocessing is preferable in terms of stabilizing the capillary flow in the channel because glass has a small contact angle with an aqueous fluid.

The first and second substrates (1 and 2) are then disposed to face each other, and joined together using e.g., the solvent method to form the nano interstices (4). Nano interstices (4) of a preferred dimension may be formed by joining the first and second substrates (1 and 2) by applying an appropriate pressure thereto for a predetermined period of time, which can be conducted by any of those skilled in the art. As mentioned above, the height of the nano interstices (4) is preferably set in the range of 10 nm to 5 μm.

Also, the shape of the nano interstices (4) is not particularly limited, and any of those illustrated in FIGS. 2D to 2G may be employed.

To form the nano interstices (4), the first and second substrates are joined using at least one joining process selected from the group consisting of processes using a solvent, ultrasonic radiation, an adhesive, a tape, heat, a laser beam, and pressure application.

Examples of the joining process include processes using a solvent, heat, pressure application and a laser beam, to fuse only the peripheral regions of the two substrates so that the unjoined regions thereof form the nano interstices (4) (see FIG. 2A.) Also employables for the same purpose are a process of joining only preformed protruded portions of a substrate to another substrate using ultrasonic radiation such that the inner region of the joined region of the joined substrate serves as the nano interstices (4) (see FIG. 2B), and a process of joining only a predetermined region of the two substrates using an adhesive or a tape such that the inner region of the substrates other than the joined region can be used as the nano interstices (4) (see FIG. 2C.)

In comparison with the conventional joining process which is performed by applying a solvent to simply perform the joining of two substrates, the inventive joining process is performed by disposing the first and second substrates to face each other, and then injecting the solvent around the periphery of the joining section of the substrate so that the injected solvent dissolves only a predetermined part of the peripheral regions of the first and second substrates, and the inner part thereof left undissolved serves as the nano interstices. Alternatively, the inventive joining process may be performed by disposing the first and second substrates to face each other and then joining only peripheral regions of the substrates using heat or a laser, instead of joining the entire contact region therebetween so that the unjoined region of the interface between the first and second substrates is used as the nano interstices.

According to the inventive joining process, the microfluidic channel having nano interstices can be formed using a single continuous process and the height of the microfluidic channel can be precisely controlled.

In the present invention, the nano interstices may be formed during or after joining of the first and second substrates, or alternatively, it may be preformed in the first or second substrate before joining of the first and second substrates, wherein the shape of the nano interstices may be easily adjusted depending on the structure of the microfluidic channel.

As mentioned above, the nano interstices can be formed without the need to add additional steps to the conventional preparation of the microfluidic channel (see FIG. 1A.) The nano interstices of the present invention may be provided by slightly changing the conventional preparation process.

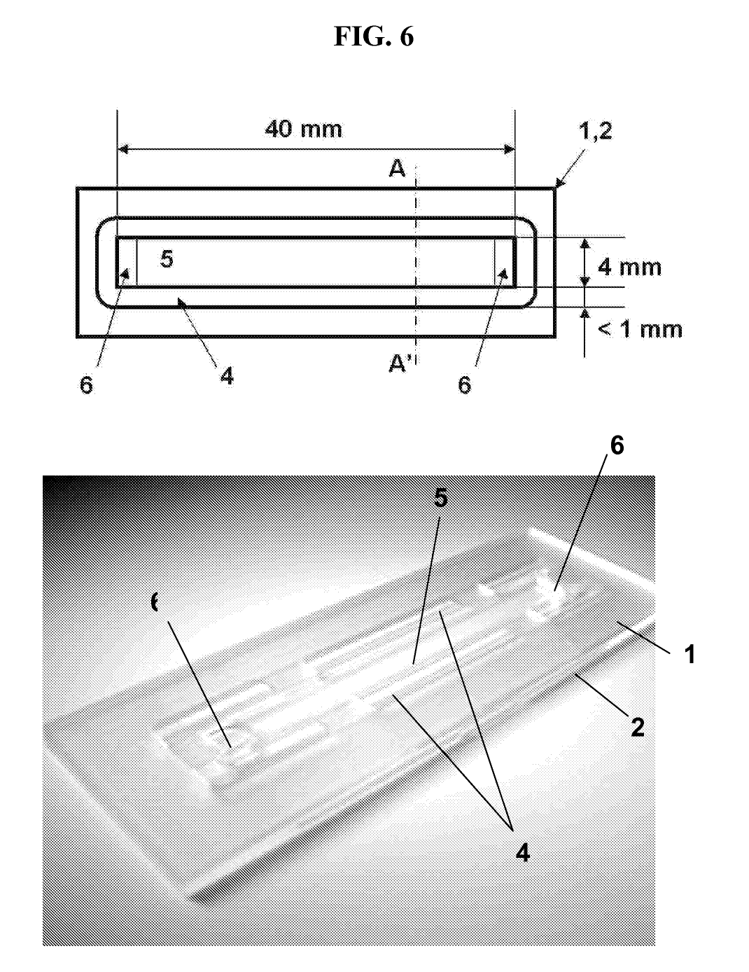

FIG. 6 shows the microfluidic circuit element having an inlet or outlet (6) for a sample to be analyzed and diagnosed, according to the embodiment of the present invention, and a photograph thereof.

The sample which can be analyzed or diagnosed using the inventive microfluidic circuit element includes any inorganic or organic sample, preferably, a biological sample such as blood, body fluid, urine or saliva. Thus, the microfluidic circuit element can be used in various applications for analysis or diagnosis of a sample and can be applied to various diagnostic kits for various diseases, e.g., a biosensor, a DNA analysis chip, a protein analysis chip and lab-on-a-chip.

As described above, the inventive microfluidic channel is fabricated to have nano interstices at both sides thereof, in which a fluid can easily infiltrate by capillary force. The fluid having infiltrated the nano interstices makes it easy to load the channel to enhance the fluid transfer therethrough. Thus, the nano interstices improve the surface wettability. Also, a stable flow of the fluid can be achieved even without any surface treatment of the channel for reducing the contact angle which tends to deform after long-term used or storage.

### EXAMPLE

The following examples are intended to illustrate the present invention, however these examples are not to be construed to limit the scope of the invention.

### Example 1

### Fabrication of Microfluidic Circuit Element using Inventive Joining Process

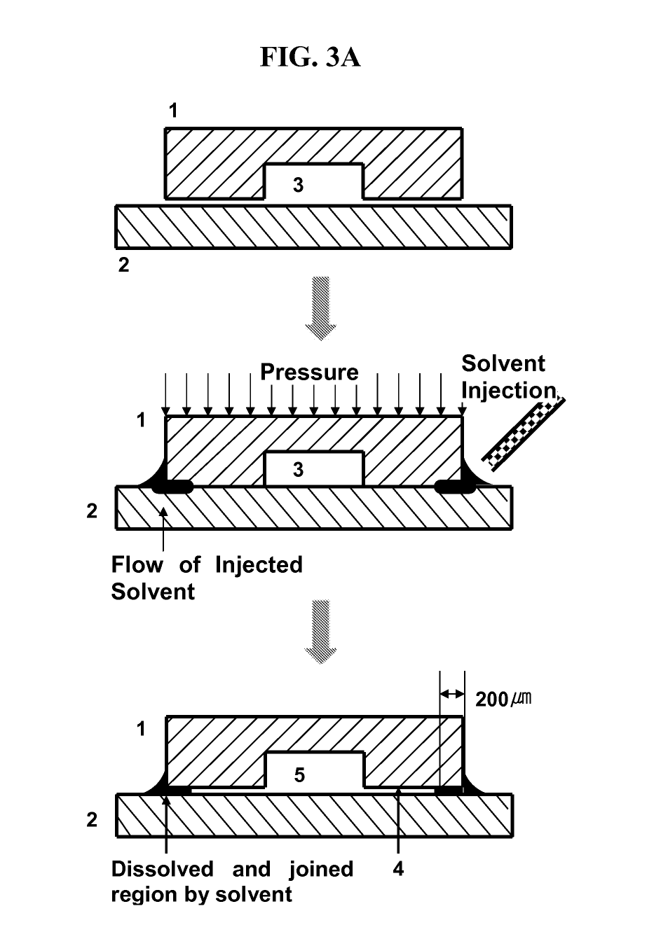

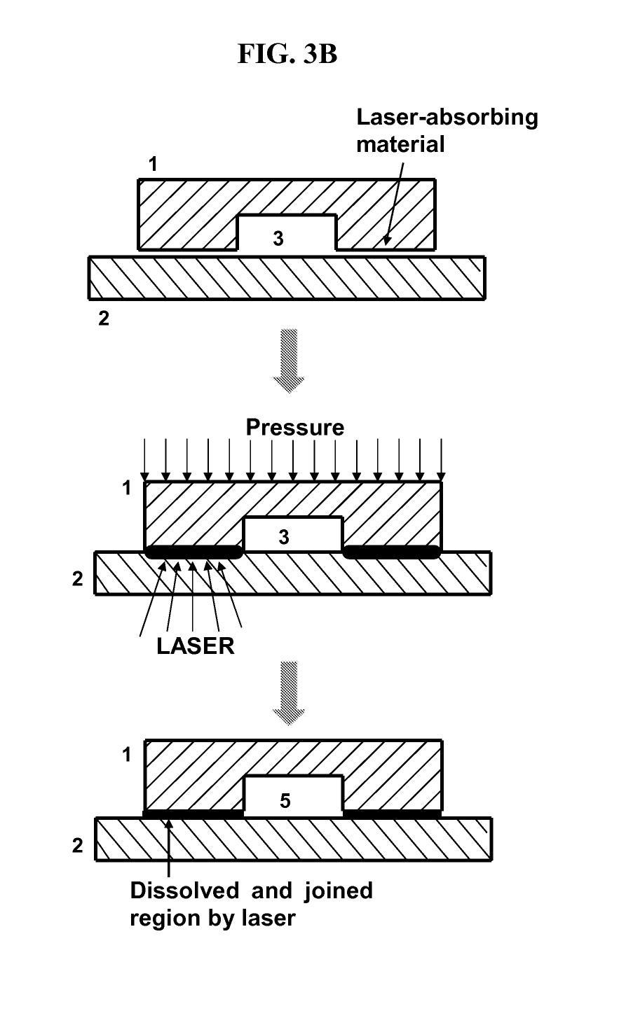

A plastic microfluidic device capable of two substrates was fabricated of PMMA (poly(methylmethacrylate)) by injection molding. Referring to FIGS. 2A and 3A, a groove (3) which would function as a channel having a rectangular cross sectional shape (a width of 4 mm, a height of 0.1 mm and a length of 40 mm) equipped with an inlet and an outlet was formed in an upper substrate (1), while a 1 mm thick lower substrate (2) having a flat surface was prepared.

The substrates (1 and 2) thus prepared by injection molding were washed with a detergent, sonicated with deionized water, dried in an oven at 60° C. overnight, and then subjected to oxygen plasma treatment for 2 min using a plasma cleaning system (available from Jesagi Hankook Ltd., Korea.)

Subsequently, the treated substrates (1 and 2) were subjected to solvent-joining to form a microfluidic channel (5) having nano interstices (4) formed therein. That is, the substrates (1 and 2) were compressed together to form an assembly having a minute space layer therebetween.

The assembly thus attained was treated with acetone injected at around the joint section as shown in the FIGS. 2A and 2B to allow the injected acetone infiltrate and dissolve a part of the interface along the length of the substrates, to join the substrates together. The applied pressure was released within 10 seconds so that the inside regions of the space between the substrates remain unjoined to form nano interstices (4) at both sides of the microfluidic channel (5) (FIG. 3A.)

The height of the interstices was controlled by adjusting the pressure-application time after the solvent injection. If the application time is reduced, the height of the nano interstices becomes higher. In the present example, the pressure-application time required to fabricate the element having the nano interstices was seven seconds. The width of the nano interstice (4) was determined by subtracting the infiltrated solvent width of about 200 μm from the initial channel wall width of 1 mm, as shown in FIG. 3A. The depth of the solvent infiltration is not dependent on the pressure-application time or the amount of solvent used, but it depends primarily on the rate at which the solvent dissolves the plastic.

The solvent joining procedure of the present example can maintain the height of the formed channel unchanged over a long-period of use. In fact, the height of the microfluidic channel (5) was measured every month over 1 year in accordance with Guidelines for Quality Assurance, and the height of the microfluidic channel (5) among 100 samples selected every month fell within 98˜102 μm.

### Comparative Example 1

### Fabrication of Microfluidic Circuit Element using Typical Joining Process

A microfluidic circuit element was formed in the same manner as in Example 1, with the exception that the entire contact surfaces of the upper and lower substrates (1 and 2) were joined using a typical laser joining process (FIG. 3B.)

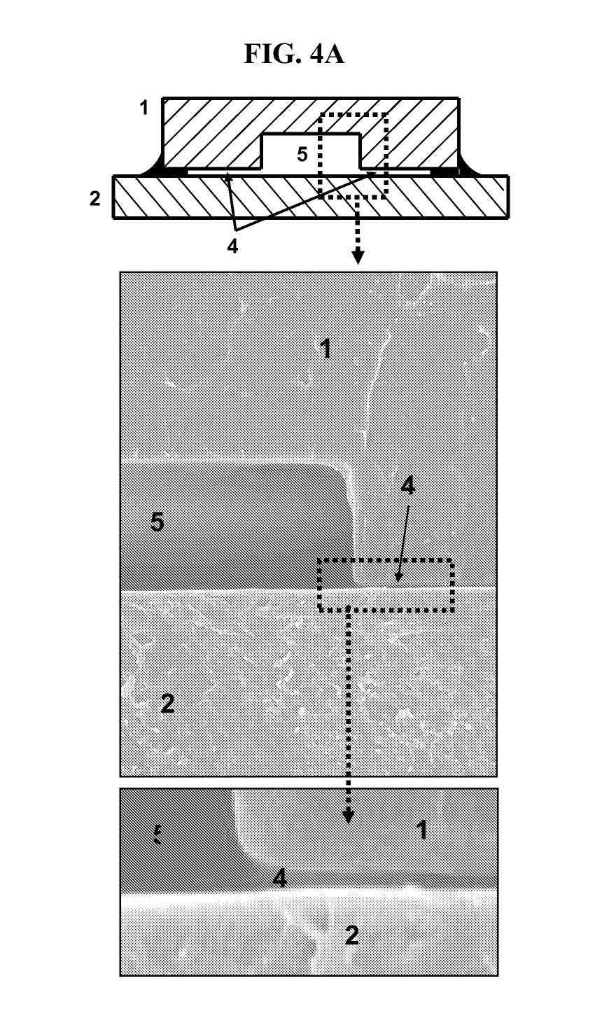

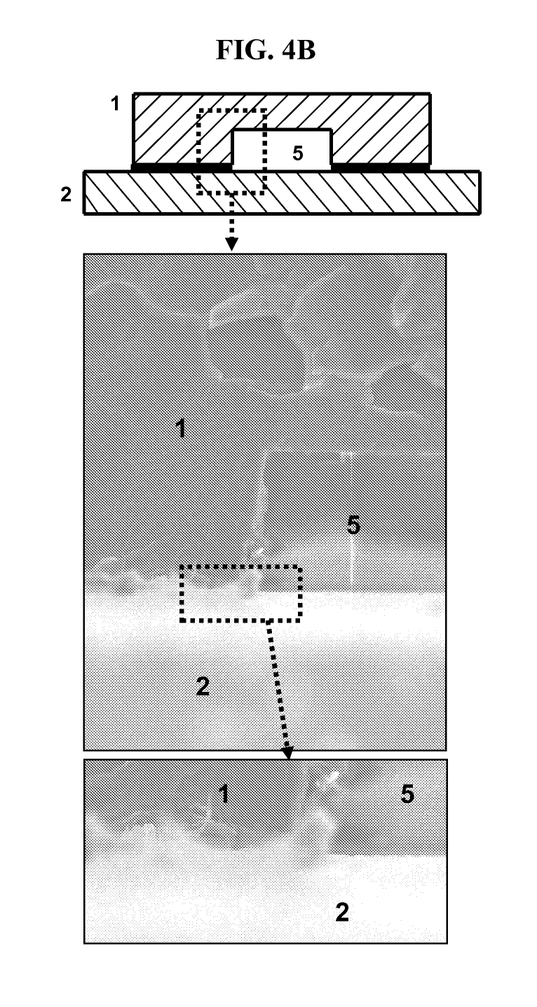

To compare the microfluidic circuit elements of Example 1 and Comparative Example 1, SEM images thereof are shown in FIGS. 4A and 4B. As shown in FIGS. 4A and 4B, the microfluidic channel (5) of Example 1 had nano interstices (4) formed at both sides thereof defined inside the peripheral joined region, whereas the microfluidic channel (5) of Comparative Example 1 had no nano interstices.

### Test 1. Evaluation of Flow Stability of Fluid

The flow of the fluid in the microfluidic channel was measured shortly after fabrication and after being stored for one year in a plastic bag, by measuring the degree of displacement (S—S₀) of the air in the interface with water in the microfluidic channel using deionized water containing a food color.

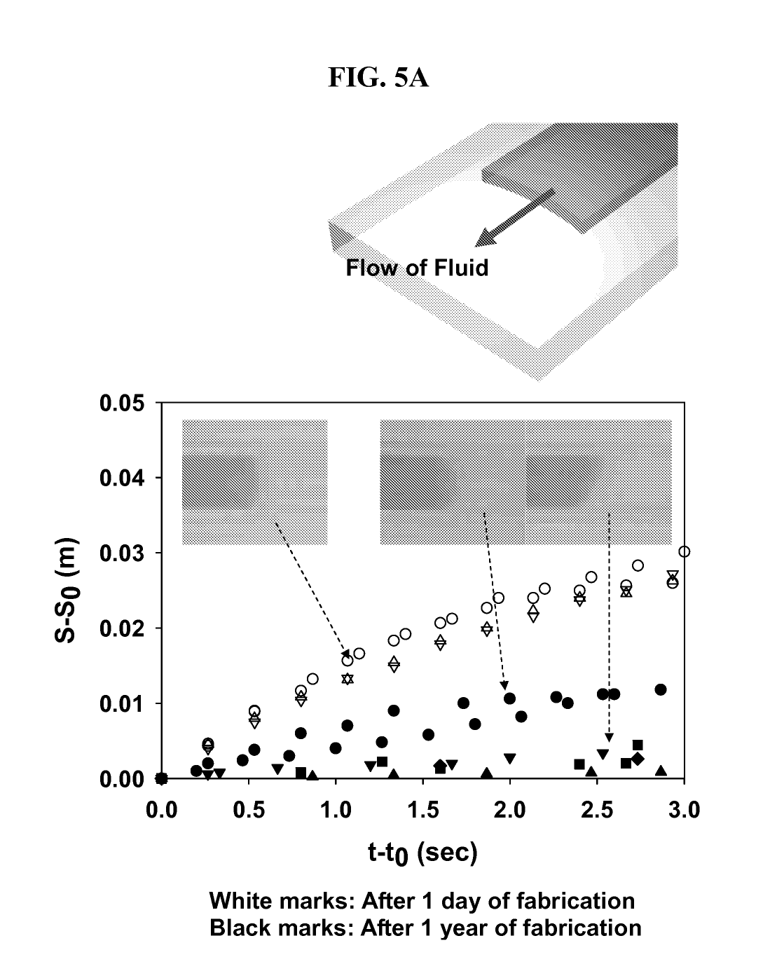

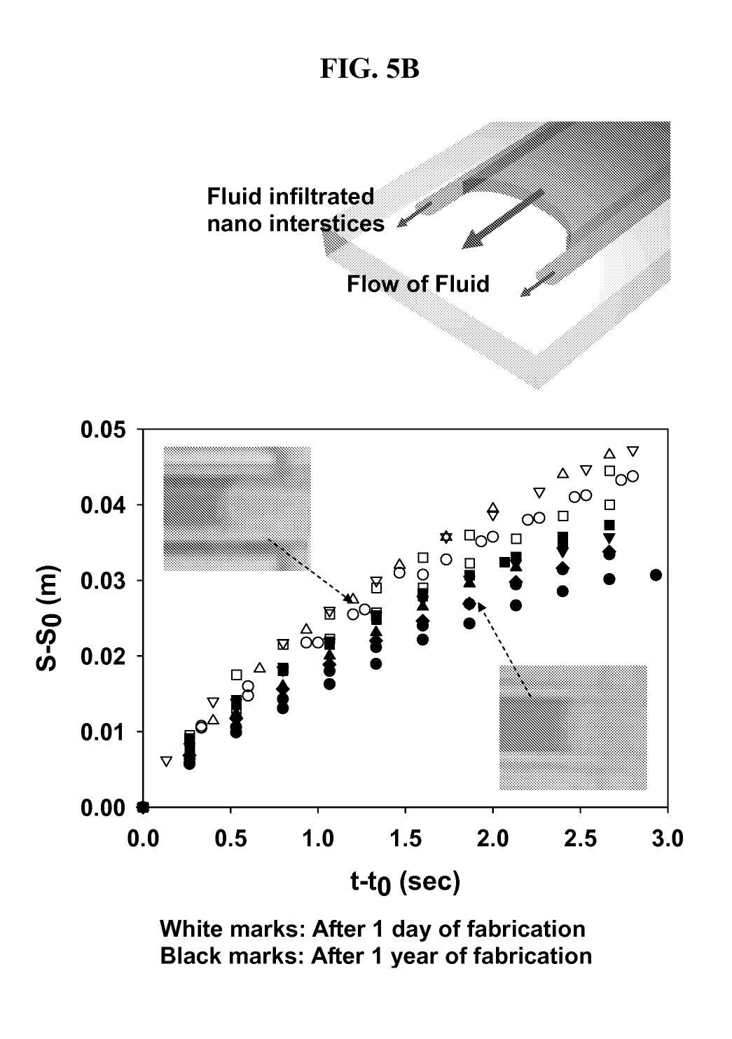

20 μl of deionized water containing a food color was introduced to the inlet of the element, the flow image thereof was photographed with a digital camera and the length of the water plug in the channel in the captured image was measured with a ruler. The results are shown in FIGS. 5A and 5B, which respectively represent the case having no nano interstices and the inventive case having nano interstices.

Referring to FIG. 5A, a stable flow of the fluid shortly after fabrication is shown (white marks.) However, after being stored for one year (black marks), almost no flow is observable. Whereas, referring to FIG. 5B, a stable flow was observed both shortly after fabrication (white marks) and after being stored for one year (black marks.) Also, the rate of the stable flow observed in the latter case was higher than that observed for the case in which the channel was not equipped with nano interstices, regardless whether the rate was measured shortly after fabrication or after being stored for one year.

While the invention has been described with respect to the above specific embodiments, it should be recognized that various modifications and changes may be made to the invention by those skilled in the art which also fall within the scope of the invention as defined by the appended claims.

1. A microfluidic circuit element, comprising a first substrate and a second substrate in a laminate form, the first substrate having a groove for defining a microfluidic channel which is formed on the side facing the second substrate and has an inlet and an outlet for a sample to flow through said channel, wherein the microfluidic channel has nano interstices formed at both sides thereof, the height of the nano interstices being less than that of the center of the microfluidic channel.

2. The microfluidic circuit element of claim 1, wherein the nano interstices are each formed at a height ranging from 10 nm to 5 μm.

3. The microfluidic circuit element of claim 1, which is used for analysis and diagnosis of a biological sample.

4. The microfluidic circuit element of claim 1, which is used as a biosensor, a DNA analysis chip, a protein analysis chip, or a lab-on-a-chip.

5. A method for fabricating a microfluidic circuit element, comprising joining a first substrate and a second substrate such that a groove is formed therebetween to serve as a microfluidic channel which has an inlet and an outlet for a sample to flow therethrough and nano interstices having a height which is less than that of the center of the microfluidic channel formed at both sides of the microfluidic channel.

6. The method of claim 5, wherein the nano interstices are formed in the first or second substrate before joining the first and second substrates, or are formed after joining the first and second substrates.

7. The method of claim 5, which further comprises subjecting at least one surface of the first and second substrates to chemical treatment or oxygen plasma treatment before joining the first and second substrates.

8. The method of claim 5, wherein the joining of the first and second substrates is performed using at least one process selected from the group consisting of processes using a solvent, ultrasonic radiation, an adhesive, a tape, heat, a laser beam and pressure application.

9. The method of any one of claim 5, wherein each of the nano interstices is formed at a height ranging from 10 nm to 5 μm.

10. The microfluidic circuit element of claim 3 , which is used as a biosensor, a DNA analysis chip, a protein analysis chip, or a lab-on-a-chip.

11. The method of claim 6, wherein each of the nano interstices is formed at a height ranging from 10 nm to 5 μm.

12. The method of claim 7, wherein each of the nano interstices is formed at a height ranging from 10 nm to 5 μm.

13. The method of claim 8, wherein each of the nano interstices is formed at a height ranging from 10 nm to 5 μm.
